(19) United States
(12) Patent Application Publication
(10) Pub. No.: US 2023/0295442 A1
(43) Pub. Date: Sep. 21, 2023

(54) PARTICLE HAVING SPECIFIC LOWER ORDER TITANIUM OXIDE CRYSTAL COMPOSITION, AND METHOD FOR PRODUCING SAME

(71) Applicant: DENKA COMPANY LIMITED, Tokyo (JP)

(72) Inventors: Takuji KOBAYASHI, Tokyo (JP); Motoharu FUKAZAWA, Tokyo (JP); Takuto OKABE, Tokyo (JP)

(73) Assignee: DENKA COMPANY LIMITED, Tokyo (JP)

(21) Appl. No.: 18/020,182

(22) PCT Filed: Aug. 13, 2021
(86) PCT No.: PCT/JP2021/029842

§ 371 Date: Feb. 7, 2023

(30) Foreign Application Priority Data
Aug. 21, 2020 (JP) 2020-140192

(51) Int. Cl. C09C 1/36

(52) U.S. Cl. CPC C09C 1/36; C01P 2006/63; C01P 2006/64; C01P 2006/80; C01P 2002/72

(57) ABSTRACT

A method for producing a particle, containing a step of heating a mixture containing TiH₂ and TiO₂ at 700 to 900° C., wherein a molar ratio of the TiH₂ to the TiO₂ contained in the mixture is 3.1 to 4.6. A particle having a crystal composition composed of Ti₂O₃ and γ-Ti₃O₅, wherein a molar ratio of the Ti₂O₃ to the γ-Ti₃O₅ is 0.1 or more.

### TECHNICAL FIELD

The present disclosure relates to a particle having a crystalline composition of Ti₂O₃ and γ-Ti₃O₅ and a method for producing the same.

### BACKGROUND ART

It is known that a low-order titanium oxide (also referred to as reduced titanium oxide) obtained by reducing titanium dioxide shows different colors depending on the ratio of titanium and oxygen which are constituent elements, and becomes black by appropriately adjusting the ratio. Therefore, particles whose surfaces are composed of a low-order titanium oxide can be used in various applications as a pigment such as a black pigment. For example, Patent Document 1 discloses a cosmetic using a pigment exhibiting dichroism in which the color tone of the appearance color and the interference color are different from each other by forming a single layer of low-order titanium oxide on plate-like particles. In addition, Patent Document 2 discloses a black titanium dioxide powder produced using CaH₂ as a reducing agent for use as a black pigment or the like. Patent Document 3 discloses titanium oxynitride powder produced by reacting titanium oxide with high-temperature ammonia gas.

### CITATION LIST

### Patent Document

Patent Document 1: Japanese Patent Application Publication No. 2010-280607
Patent Document 2: Japanese Patent Application Publication No. 2012-214348
Patent Document 3: Japanese Patent Application Publication No. 2010-30842

### SUMMARY OF INVENTION

### Technical Problem

The black pigment containing the low-order titanium oxide exhibits black colors of different colors, such as a reddish black color and a bluish black color, even if it is said to be entirely black. The color tone of black may change not only depending on the composition of the low-order titanium oxide as described above but also depending on the particle diameter of the pigment (particle) or the like. Therefore, in order to obtain a black pigment having a desired color tone, physical properties such as particle diameter may be adjusted. However, since such physical properties may be restricted by, for example, the use of the black pigment, it is preferable to obtain a black color having a desired tint only by adjusting the composition of the low-order titanium oxide.

Therefore, an object of one aspect of the present invention is to obtain a particle of a low-order titanium oxide having a novel composition.

### Solution to Problem

The present inventors have found that when a particle containing low-order titanium oxides are produced by heating TiH₂ and TiO₂, a particle having a novel composition of low-order titanium oxides can be obtained by appropriately adjusting the mixing ratio of TiH₂ and TiO₂ and the heating temperatures. The particle has a crystalline composition composed of the specific proportions of Ti₂O₃ and γ-Ti₃O₅.

That is, one aspect of the present invention is a method for producing a particle, containing a step of heating a mixture containing TiH₂ and TiO₂ at 700 to 900° C., wherein a molar ratio of TiH₂ to TiO₂ contained in the mixture is 3.1 to 4.6. In this step, the mixture may be heated under an Ar gas atmosphere.

Another aspect of the present invention is a particle having a crystal composition composed of Ti₂O₃ and γ-Ti₃O₅, wherein a molar ratio of the Ti₂O₃ to the γ-Ti₃O₅ is 0.1 or more. The particle may have a* value of 0 or more and b* value of 0 or less in L*a*b* color space. A total content of Na, K, and P in the particle may be 2000 ppm by mass or less.

Another aspect of the present invention is a dispersion containing the above-described particle and a dispersion medium.

### Advantageous Effects of Invention

According to an aspect of the present disclosure, a particle of a low-order titanium oxide having a novel composition can be obtained. This makes it easy to adjust the black color of a dispersion containing the particle of low-order titanium oxide (for example, a resin composition containing the particle of low-order titanium oxide and a resin).

### BRIEF DESCRIPTION OF DRAWINGS

FIG. 1 shows measurement results of X-ray diffraction in Examples and Comparative Examples.

FIG. 2 shows measurement results of X-ray diffraction in Examples and Comparative Examples.

FIG. 3 shows measurement results of X-ray diffraction in Examples and Comparative Examples.

### DESCRIPTION OF EMBODIMENTS

One embodiment of the present invention is a method for producing a particle (hereinafter also referred to as “low-order titanium oxide particle”) having a specific crystal composition composed of Ti₂O₃ and γ-Ti₃O₅ (details will be described later). This method contains a step of heating a mixture containing TiH₂ and TiO₂ (heating step).

The mixture used in the heating step contains, for example, a powdered TiH₂ and a powdered TiO₂. The mixture may be, for example, a powder that is not formed into a pellet shape or the like (that contains powdered TiH₂ and TiO₂ as they are). The properties of the powdered TiH₂ and TiO₂ can be selected as appropriate. For example, the particle sizes of the powdered TiH₂ and TiO₂ are selected in accordance with the desired particle size of the low-order titanium oxide particle. The mixture may consist of TiH₂ and TiO₂, or may consist of TiH₂, TiO₂, and unavoidable impurities. Examples of the unavoidable impurities include Al₂O₃, ZrO₂, and C (carbon). The total amount of TiH₂ and TiO₂ in the mixture may be 90% by mass or more, 95% by mass or more, or 99% by mass or more, based on the total amount of the mixture.

The molar ratio of TiH₂ to TiO₂ contained in the mixture (content of TiO₂ (mol)/content of TiH₂ (mol)) is 3.1 to 4.6. When the molar ratio is less than 3.1, γ-Ti₃O₅ is not formed in the obtained particle. In this case, the low-order titanium oxide particle tend to exhibit a black-yellow color. When the molar ratio is more than 4.6, Ti₂O₃ is not formed in the obtained particle. In this case, the low-order titanium oxide particle tends to exhibit a black-blue color.

The larger the molar ratio, the higher the proportion of γ-Ti₃O₅ in the resulting particle and the lower the proportion of Ti₂O₃. The lower limit of the molar ratio may be 3.2 or more, 3.3 or more, 3.4 or more, 3.5 or more, 3.6 or more, 3.7 or more, 3.8 or more, 3.9 or more, 4.0 or more, 4.1 or more, or 4.2 or more. The upper limit of the molar ratio may be 4.5, 4.4, 4.3, 4.2, 4.1, 4.0, 3.9, 3.8, 3.7, 3.6, or 3.5.

In the heating step, the mixture is heated at 700 to 900° C. in, for example, an electric furnace. As a result, the titanium dioxide is reduced to produce desired low-order titanium oxides (Ti₂O₃ and γ-Ti₃O₅) in the resulting particle. When the heating temperature is lower than 700° C., Ti₂O₃ and γ-Ti₃O₅ are not generated in the obtained particle, and for example, TiₙO₂ₙ₋₁ (n>4) may be generated. When the heating temperature is higher than 900° C., γ-Ti₃O₅ may not be produced in the obtained particle, and for example, α-Ti₃O₅ and β-Ti₃O₅ may be produced.

The mixture is heated, for example, under an inert gas atmosphere. The inert gas may be an Ar gas or a N₂ gas, and is preferably an Ar gas from the viewpoint that the low-order titanium oxide particle having a desired crystal composition can be more easily obtained (for example, generation of TiOₓ (x≥1.75) in the low-order titanium oxide particle can be further suppressed).

The heating time may be, for example, 1 hour or more, 2 hours or more, or 4 hours or more, from the viewpoint of sufficiently proceeding the reduction reaction, and may be, for example, 24 hours or less, 18 hours or less, or 12 hours or less, from the viewpoint of appropriately suppressing the growth of the lower-order titanium oxide particle and easily recovering the particle in a powder state.

In one embodiment, the method may further contain a step of washing the low-order titanium oxide particle (washing step). Impurities can be removed by the washing step. The washing is performed with, for example, at least one selected from the group consisting of a hot water, an alcohol, and an organic acid. The alcohol may be, for example, methanol, ethanol, or mixtures thereof. The organic acid may be, for example, acetic acid. From the viewpoint of being able to suppress mixing of ionic impurities such as halide ions into the low-order titanium oxide powder, washing with an organic acid is preferable.

The method preferably further contains a step of pulverizing the low-order titanium oxide particle after the heating step (pulverizing step). Examples of the pulverizing method in the pulverizing step include methods using various pulverizers such as a mortar, a ball mill, a jet mill, and a fine mill. The pulverizing step may be performed once or two or more times. When the pulverizing step is performed two or more times, the pulverizing method used in each pulverizing step may be different from each other. By performing the pulverizing step, the chromaticity and the specific surface area of the low-order titanium oxide particle can be adjusted.

When the method contains the washing step and the pulverizing step, the order of these steps is arbitrary. That is, the method may contain the heating step, the washing step, and the pulverizing step in this order, or may contain the heating step, the pulverizing step, and the washing step in this order. In the former case, a step of drying the low-order titanium oxide particle (drying step) may be further performed between the washing step and the pulverizing step. The drying temperature in the drying step may be, for example, 100° C. or higher and 200° C. or lower. The drying time may be, for example, 10 hours or more and 20 hours or less.

The low-order titanium oxide particle obtained by the production method described above has a crystal composition composed of Ti₂O₃ and γ-Ti₃O₅. The crystal composition composed of Ti₂O₃ and γ-Ti₃O₅ means that the crystal composition substantially consisting of Ti₂O₃ and γ-Ti₃O₅. It is confirmed that the low-order titanium oxide particle has a crystal composition composed of Ti₂O₃ and γ-Ti₃O₅, by measuring the crystal composition of the low-order titanium oxide particle by an X-ray diffraction method (XRD) and substantially observing only diffraction peaks derived from Ti₂O₃ and γ-Ti₃O₅. The low-order titanium oxide particle may be composed of a mixed phase of two crystal phases of Ti₂O₃ and γ-Ti₃O₅ in a single particle.

In the crystal composition of the low-order titanium oxide particle, the molar ratio of Ti₂O₃ to γ-Ti₃O₅ (content of γ-Ti₃O₅ (mol)/content of Ti₂O₃ (mol)) is 0.1 or more. The molar ratio may be 0.2 or more, 0.3 or more, 0.4 or more, 0.5 or more, 0.6 or more, 0.7 or more, 0.8 or more, 0.9 or more, or 1.0 or more, and may be 50 or less, 40 or less, 30 or less, 25 or less, 20 or less, 15 or less, 10 or less, 8 or less, or 5 or less. The molar ratio is calculated by the following formula:

Molar ratio(γ-Ti₃O₅/Ti₂O₃)=(M1/F1)/(M2/F2)

wherein M1 represents the mass fraction of γ-Ti₃O₅ in the low-order titanium oxide particle, F1 represents the formula mass of γ-Ti₃O₅ (=223.60), M2 represents the mass fraction of Ti₂O₃ in the low-order titanium oxide particle, and F2 represents the formula mass of Ti₂O₃ (=143.73).

The mass fractions of γ-Ti₃O₅ (M1) and Ti₂O₃ (M2) in the low-order titanium oxide particle are calculated by Rietveld analysis of the X-ray diffraction pattern. To be specific, the mass fractions are calculated by using Rietveld method software (for example, integrated powder X-ray analysis software PDXL2 manufactured by Rigaku Corporation), and by using 1243140 (Journal of Applied Physics 119, 014905 (2016)) as Ti₂O₃ and 1900755 (Journal of Solid State Chemistry 20, 29 (1977)) as γ-Ti₃O₅ from a crystal structure database (Pearson's Crystal Data).

The low-order titanium oxide particle has the above-described crystal composition and thus exhibit a black color having a specific chromaticity. The L* value of the low-order titanium oxide particle in the L*a*b* color space is preferably 13 or less, more preferably 11 or less, and even more preferably 10 or less, and may be, for example, 4 or more, 5 or more, or 6 or more. The a* value of the low-order titanium oxide particle in the L*a*b* color space is preferably −1 or more, more preferably 0 or more, and preferably 8 or less, more preferably 6 or less, even more preferably 4 or less. The b* value of the low-order titanium oxide particle in the L*a*b* color space is preferably −8 or more, more preferably −6 or more, and even more preferably −4 or more, and is preferably 1 or less, and more preferably 0 or less.

The L* value, the a* value, and the b* value in the L*a*b* color space are measured by a colorimetric color difference meter (for example, ZE-2000 (manufactured by Nippon Denshoku Industries Co., Ltd.)). More specifically, after zero point correction is performed with a cylinder for dark field, standard matching is performed with a standard white plate (X=91.71, Y=93.56, Z=110.52). Next, about 3 g of the low-order titanium oxide particles are put into a round cell of 35φ×15H, and measurement is performed.

The specific surface area of the low-order titanium oxide particle may be 0.25 m²/g or more, 1 m²/g or more, 2 m²/g or more, 3 m²/g or more, or 4 m²/g or more, and may be 20 m²/g or less, 10 m²/g or less, or 8 m²/g or less. The specific surface area of the low-order titanium oxide particle is measured using a specific surface area measuring device (for example, Macsorb HM model-1201, manufactured by Mountech Co., Ltd.) under the conditions in which degassing is performed at 200° C. for 10 minutes with a nitrogen gas flow (atmospheric pressure), and nitrogen gas adsorption is performed at an equilibrium relative pressure of about 0.3 under n=2 conditions.

The contents of impurities in the low-order titanium oxide particle are preferably as small as possible. The content of A1 in the low-order titanium oxide particle may be preferably 200 ppm by mass or less, 50 ppm by mass or less, or 20 ppm by mass or less. The content of B in the low-order titanium oxide particle may be preferably 50 ppm by mass or less, 30 ppm by mass or less, or 10 ppm by mass or less. The content of Ba in the low-order titanium oxide particle may be preferably 50 ppm by mass or less, 10 ppm by mass or less, or 5 ppm by mass or less. The content of Ca in the low-order titanium oxide particle may be preferably 100 ppm by mass or less, 50 ppm by mass or less, or 10 ppm by mass or less. The content of Cd in the lower-order titanium oxide particle may be preferably 10 ppm by mass or less, 5 ppm by mass or less, or 2 ppm by mass or less. The content of Co in the lower-order titanium oxide particle may be preferably 10 ppm by mass or less, 5 ppm by mass or less, or 2 ppm by mass or less. The content of Cr in the low-order titanium oxide particle may be preferably 100 ppm by mass or less, 10 ppm by mass or less, or 5 ppm by mass or less. The content of Cu in the low-order titanium oxide particle may be preferably 200 ppm by mass or less, 50 ppm by mass or less, or 10 ppm by mass or less. The content of Fe in the low-order titanium oxide particle may be preferably 200 ppm by mass or less, 50 ppm by mass or less, or 10 ppm by mass or less. The content of K in the low-order titanium oxide particle may be preferably 100 ppm by mass or less, 5 ppm by mass or less, or 1 ppm by mass or less. The content of Li in the lower-order titanium oxide particle may be preferably 20 ppm by mass or less, 2 ppm by mass or less, or 0.5 ppm by mass or less.

The content of Mg in the lower-order titanium oxide particle may be preferably 100 ppm by mass or less, 10 ppm by mass or less, or 1 ppm by mass or less. The content of Mn in the low-order titanium oxide particle may be preferably 10 ppm by mass or less, 5 ppm by mass or less, or 2 ppm by mass or less. The content of Mo in the low-order titanium oxide particle may be preferably 10 ppm by mass or less, 5 ppm by mass or less, or 2 ppm by mass or less. The content of Na in the low-order titanium oxide particle may be preferably 50 ppm by mass or less, 5 ppm by mass or less, or 2 ppm by mass or less. The content of Ni in the low-order titanium oxide particle may be preferably 50 ppm by mass or less, 20 ppm by mass or less, or 10 ppm by mass or less. The content of P in the low-order titanium oxide particle may be preferably 200 ppm by mass or less, 30 ppm by mass or less, or 5 ppm by mass or less. The content of Pb in the low-order titanium oxide particle may be preferably 50 ppm by mass or less, 5 ppm by mass or less, or 2 ppm by mass or less. The content of Sb in the low-order titanium oxide particle may be preferably 100 ppm by mass or less, 10 ppm by mass or less, or 2 ppm by mass or less. The content of Si in the low-order titanium oxide particle may be preferably 1000 ppm by mass or less, 100 ppm by mass or less, 30 ppm by mass or less, or 2 ppm by mass or less. The content of Zn in the lower-order titanium oxide particle may be preferably 100 ppm by mass or less, 10 ppm by mass or less, or 2 ppm by mass or less. The content of Zr in the lower-order titanium oxide particle may be preferably 100 ppm by mass or less, 20 ppm by mass or less, or 2 ppm by mass or less.

The total content of Na, K, and Pin the lower-order titanium oxide particle may be preferably 2000 ppm by mass or less, 1000 ppm by mass or less, 500 ppm by mass or less, or 100 ppm by mass or less. The total content of Pb, Cd, and Cr in the lower-order titanium oxide particle may be preferably 200 ppm by mass or less, 100 ppm by mass or less, 50 ppm by mass or less, or 30 ppm by mass or less. The contents of impurities in the low-order titanium oxide particle are measured by elemental analysis (for example, Agilent 5110ICP-OES (manufactured by Agilent Technologies, Inc.)).

The above-described low-order titanium oxide particle is suitably used as a pigment (colored filler) such as a black pigment. Such a pigment (colored filler) is suitably used as a coloring agent including, for example, cosmetics, electronic components such as semiconductors, and paints such as paints and inks.

When the low-order titanium oxide particles are used in the above-described application, the low-order titanium oxide particles are used by being dispersed in a dispersion medium, for example. That is, another embodiment of the present invention is a dispersion containing the above-described low-order titanium oxide particles and a dispersion medium dispersing the low-order titanium oxide particles.

The dispersion medium is appropriately selected depending on the application of the dispersion, and may be, for example, water, an alcohol, a ketone, an ester, a resin, or the like. Examples of the resin include an epoxy resin, a silicone resin, a phenol resin, a melamine resin, an urea resin, an unsaturated polyester, a fluororesin, a polyimide, a polyamideimide, a polyetherimide, a polybutylene terephthalate, a polyethylene terephthalate, a polyphenylene sulfide, a wholly aromatic polyester, a polysulfone, a liquid crystal polymer, a polyethersulfone, a polycarbonate, a maleimide-modified resin, an ABS (acrylonitrile butadiene styrene) resin, an AAS (acrylonitrile acrylic rubber styrene) resin, an AES (acrylonitrile ethylene propylene diene rubber styrene) resin, and the like.

The content of the low-order titanium oxide particles in the dispersion is appropriately selected depending on the application of the dispersion, and, for example, may be 5% by mass or more and may be 90% by mass or less, based on the total amount of the dispersion. The content of the dispersion medium in the dispersion is appropriately selected depending on the application of the dispersion, and, for example, may be 10% by mass or more and may be 95% by mass or less, based on the total amount of the dispersion.

### EXAMPLES

Hereinafter, the present invention will be described in more detail based on examples, but the present invention is not limited to the following examples.

<Production of Low-Order Titanium Oxide Particle>

### Example 1

10 g of TiO₂ powder (manufactured by Toho Titanium, HT0514: purelity of 99.9%) and 2.02 g of TiH₂ powder (manufactured by Tohotech, TCH450: purelity of 99.8%) were mixed (TiO₂/TiH₂=3.1/1 (molar ratio)) in an Eirich mixer (manufactured by Nippon Eirich Co., Ltd.) to obtain a mixture. This mixture was transferred to an alumina crucible and heated for 12 hours in an electric furnace (HIMULTI 5000 manufactured by Fuji Electric Wave Industry Co., Ltd.) in a state in which the temperature was raised to 800° C. at 10° C./min under an Ar atmosphere. After heating, the obtained powder was pulverized in a mortar for 5 minutes to obtain black low-order titanium oxide particles.

### Examples 2 to 9

Black low-order titanium oxide particles were obtained in the same manner as in Example 1 except that the amount of the powder of TiH₂ was changed so that the molar ratio of TiO₂ to TiH₂ (TiO₂/TiH₂) was as shown in Table 1.

### Example 10

Black low-order titanium oxide particles were obtained in the same manner as in Example 6 except that the heating time was changed to 4 hours.

### Examples 11 and 12

Black low-order titanium oxide particles were obtained in the same manner as in Example 6 except that the heating temperature was changed as shown in Table 1.

### Comparative Examples 1 and 2

Particles were obtained in the same manner as in Example 1 except that the amount of the powder of TiH₂ was changed so that the molar ratio of TiO₂ to TiH₂ (TiO₂/TiH₂) was as shown in Table 1.

### Comparative Examples 3 and 4

Particles were obtained in the same manner as in Example 6 except that the heating temperature was changed as shown in Table 1.

<X-Ray Diffraction Measurement>

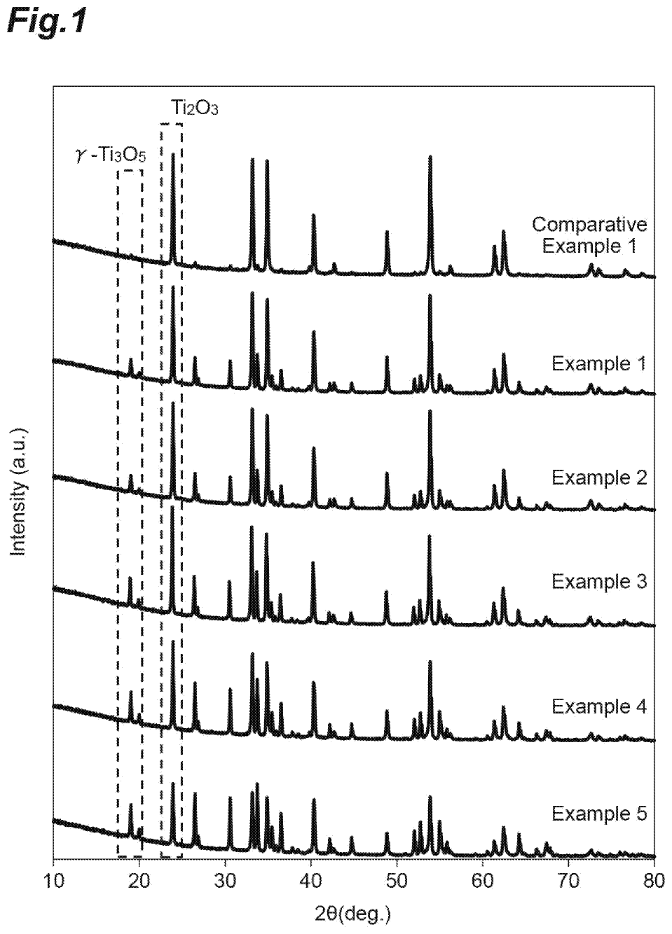

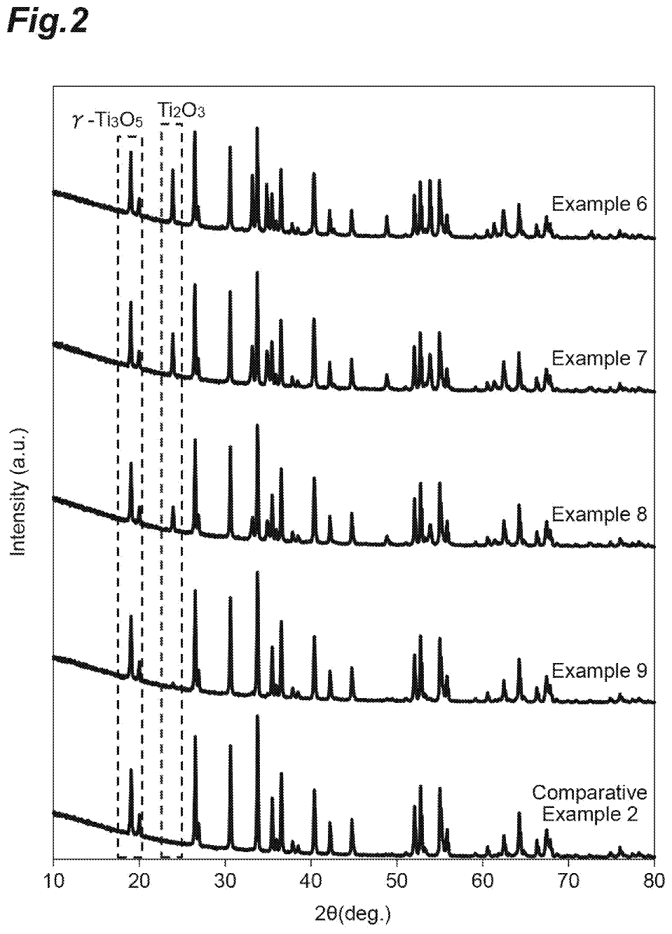

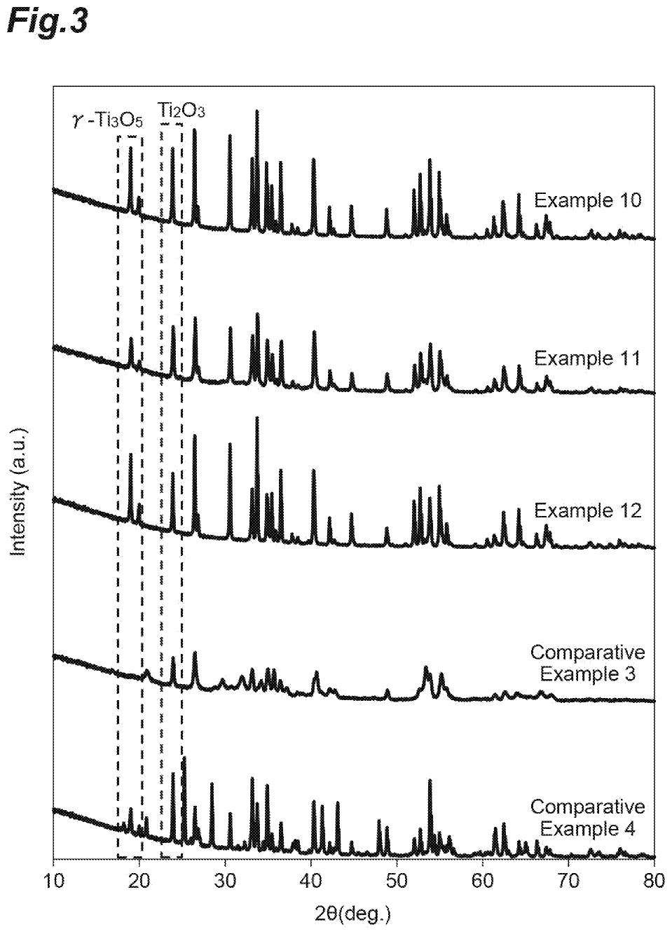

Each of the particles of Examples and Comparative Examples were subjected to powder X-ray diffraction measurement. Specifically, a horizontal sample multipurpose X-ray diffractometer (RINT-UltimaIV manufactured by Rigaku Corporation) was used to measure a diffraction pattern under the following measurement conditions. The obtained X-ray diffraction patterns are shown in FIGS. 1 to 3.

(Measurement Conditions)
X-ray source: Cu—K α radiation (λ=1.54184 Å)
Tube voltage: 40 kV, tube current: 40 mA
Optical conditions for measurement: divergence slit=2/3°
Scattering slit: 8 mm
Light-receiving slit=0. 15 mm
Position of diffraction peak=2θ (diffraction angle)
Scan speed: 4.0° (2θ)/min, continuous scan
Measurement range: 2θ=10° to 80°

Subsequently, the mass fractions (% by mass) of Ti₂O₃ and γ-Ti₃O₅ in the obtained particles were calculated using Rietveld method software (integrated powder X-ray analysis software PDXL2 manufactured by Rigaku Corporation). From the crystal structure database (Pearson's Crystal Data), 1243140 (Journal of Applied Physics 119, 014905 (2016)) was used as the Ti₂O₃, and 1900755 (Journal of Solid State Chemistry 20, 29 (1977)) was used as the γ-Ti₃O₅. Further, from the mass fraction M1 of γ-Ti₃O₅ and the mass fraction M2 of Ti₂O₃, and the formula mass F1 (=223.60) of γ-Ti₃O₅ and the formula mass F2 (=143.73) of Ti₂O₃, the molar ratio of γ-Ti₃O₅ to Ti₂O₃ (γ-Ti₃O₅/Ti₂O₃) is was calculated by the following formula:

Molar ratio(γ-Ti₃O₅/Ti₂O₃)=(M1/F1)/(M2/F2)

The results are shown in Table 1.

<Measurement of Chromaticity>

The chromaticity (L*value, a*value, and b*value in the L*a*b* color space) of each of the particles of Examples and Comparative Examples were measured using a colorimetric color difference meter ZE-2000 (manufactured by Nippon Denshoku Industries Co., Ltd.). More specifically, first, zero point correction was performed with a cylinder for dark field, and then standard matching was performed with a standard white plate (X=91.71, Y=93.56, Z=110.52). Next, 3 g of the particles were put into a round cell of 35φ×15 H, and the chromaticity was measured. The results are shown in Table 1.

<Measurement of Specific Surface Area>

The specific surface area of each of the particles of the above Examples was measured using a specific surface area measuring device (Macsorb HM model-1201, manufactured by Mountech Co.). Degassing was carried out by nitrogen gas flow (atmospheric pressure) at 200° C. for 10 minutes. The measurement conditions were nitrogen gas adsorption, an equilibrium relative pressure of about 0.3, and n=2. The results are shown in Table 1.

TABLE 1

| | | | | Properties of particles | | | | | | | |
|---|---|---|---|---|---|---|---|---|---|---|---|
| | Production condition | | | | | | | | | | Specific |
| | | Heating | Heating | | Mass fraction | | Molar ratio | | | | surface |
| | Molar ratio | time | temperature | Crystal | (% by mass) | | (γ-Ti₃O₅/ | Chromaticity | | | area |
| | (TiO₂/TiH₂) | (hours) | (° C.) | composition | Ti₂O₃ | γ-Ti₃O₅ | Ti₂O₃) | L* | a* | b* | (m²/g) |
| Example | 3.1 | 12 | 800 | Ti₂O₃, | 79.7 | 20.3 | 0.121 | 10.7 | 2.7 | −0.3 | 2.6 |
| 1 | | | | γ-Ti₃O₅ | | | | | | | |
| Example | 3.2 | 12 | 800 | Ti₂O₃, | 68.6 | 31.4 | 0.294 | 10.6 | 2.4 | −0.4 | 2.4 |
| 2 | | | | γ-Ti₃O₅ | | | | | | | |
| Example | 3.4 | 12 | 800 | Ti₂O₃, | 61.9 | 38.1 | 0.396 | 10.5 | 2.1 | −0.5 | 2.2 |
| 3 | | | | γ-Ti₃O₅ | | | | | | | |
| Example | 3.6 | 12 | 800 | Ti₂O₃, | 54.2 | 45.8 | 0.543 | 10.1 | 1.6 | −0.9 | 2.3 |
| 4 | | | | γ-Ti₃O₅ | | | | | | | |
| Example | 3.8 | 12 | 800 | Ti₂O₃, | 42.7 | 57.3 | 0.863 | 10.7 | 1.1 | −1.5 | 2.3 |
| 5 | | | | γ-Ti₃O₅ | | | | | | | |
| Example | 4.0 | 12 | 800 | Ti₂O₃, | 25.1 | 74.9 | 1.92 | 9.6 | 1.0 | −1.5 | 2.4 |
| 6 | | | | γ-Ti₃O₅ | | | | | | | |
| Example | 4.2 | 12 | 800 | Ti₂O₃, | 22.9 | 77.1 | 2.16 | 10.0 | 0.8 | −1.9 | 2.5 |
| 7 | | | | γ-Ti₃O₅ | | | | | | | |
| Example | 4.4 | 12 | 800 | Ti₂O₃, | 13.9 | 86.1 | 3.98 | 10.0 | 0.5 | −2.2 | 2.4 |
| 8 | | | | γ-Ti₃O₅ | | | | | | | |
| Example | 4.6 | 12 | 800 | Ti₂O₃, | 2.2 | 97.8 | 28.6 | 10.3 | 0.1 | −2.5 | 2.5 |
| 9 | | | | γ-Ti₃O₅ | | | | | | | |
| Example | 4.0 | 4 | 800 | Ti₂O₃, | 25.1 | 74.9 | 1.92 | 9.4 | 0.5 | −1.9 | 2.4 |
| 10 | | | | γ-Ti₃O₅ | | | | | | | |
| Example | 4.0 | 12 | 700 | Ti₂O₃, | 28.4 | 71.4 | 1.62 | 10.0 | 0.6 | −2.0 | 2.5 |
| 11 | | | | γ-Ti₃O₅ | | | | | | | |
| Example | 4.0 | 12 | 900 | Ti₂O₃, | 26.1 | 73.9 | 1.82 | 10.2 | 1.0 | −1.4 | 2.4 |
| 12 | | | | γ-Ti₃O₅ | | | | | | | |
| Comp. | 3.0 | 12 | 800 | Ti₂O₃ | 100 | 0 | 0.00 | 11.2 | 2.9 | 0.2 | — |
| Example | | | | | | | | | | | |
| 1 | | | | | | | | | | | |
| Comp. | 4.8 | 12 | 800 | γ-Ti₃O₅ | 0 | 100 | — | 9.5 | -0.1 | −2.4 | — |
| Example | | | | | | | | | | | |
| 2 | | | | | | | | | | | |
| Comp. | 4.0 | 12 | 600 | TiₙO₂ₙ₋₁ | — | — | — | 23.1 | -3.2 | −6.7 | — |
| Example | | | | (n > 4) | | | | | | | |
| 3 | | | | | | | | | | | |
| Comp. | 4.0 | 12 | 1000 | Ti₂O₃, | — | — | — | 14.0 | 2.7 | 0.1 | — |
| Example | | | | α-Ti₃O₅, | | | | | | | |
| 4 | | | | β-Ti₃O₅ | | | | | | | |

### Example 13

Using a single track jet mill model FS-4 (manufactured by Seishin Enterprise Co., Ltd.), the black low-order titanium oxide particles obtained in Example 6 were pulverized under the following pulverizing conditions to obtain black low-order titanium oxide particles.

(Pulverizing Conditions)

Pressures of the pressure nozzle (pressures of air for pushing the raw material into the pulverizing chamber)/pressures of the gliding nozzle (pressures of air for hitting the raw material against each other in the pulverizing chamber): 0. 70 MPa/0. 70 MPa

Throughput: 1.5 kg/hr

### Examples 14 and 15

Using a fine mill SF15 (manufactured by Nippon Coke & Engineering Co., Ltd.), the black low-order titanium oxide particles obtained in Example 6 were pulverized and classified under the following pulverizing and classificating conditions, and the black low-order titanium oxide particles were cyclone and a bag filter in the fine mill. The black low-order titanium oxide particles collected by the cyclone were referred to as Example 14, and the black low-order titanium oxide particles collected by the bag filter were referred to as Example 15.

(Pulverizing and Classificating Conditions)
Ball: zirconia (size: φ5, amount used: 30.6 kg)
Agitator: rotation speed: 440 min⁻¹
Classifying rotor: rotation speed: 8000 min⁻¹
Roots blower: frequency: 30.0 Hz
Pulverizing aid: ethanol (0.5 wt % based on the raw material)

The chromaticity and specific surface area of each of the particles of Examples 13 to 15 were measured in the same manner as described above. The results are shown in Table 2.

TABLE 2

| | | Properties of particles | | | |
|---|---|---|---|---|---|
| | Production | | | | Specific |
| | condition | Chromaticity | | | surface area |
| | Pulverizer | L* | a* | b* | (m²/g) |
| Example | Jet mill | 10.3 | 1.4 | −1.4 | 4.5 |
| 13 | | | | | |
| Example | Fine mill | 10.6 | 1.1 | −1.4 | 3.5 |
| 14 | (collected by cyclone) | | | | |
| Example | Fine mill | 10.6 | 0.7 | −1.6 | 4.8 |
| 15 | (collected by bag filter) | | | | |

<Elemental Analysis>

Each of the particles in the above Examples was subjected to elemental analysis using Agilent 5110ICP-OES (manufactured by Agilent Technologies, Inc.). To be specific, 0.1 g of the particles were weighed in a platinum crucible, 1 ml of each of HF and HCl were added thereto, and pressure acidolysis was performed at 150° C. for 4 hours. Thereafter, the volume was fixed at 6 ml, and after confirming that there was no unnecessary residue, ICP emission spectral analysis was performed. The results are shown in Table 3. In Table 3, “ND” means that the value was equal to or less than the minimum limit of detection, and the numerical value in parentheses means that the value was equal to or less than the minimum limit of quantification. The minimum limit of detection and the minimum limit of quantification are as follows.

(Minimum Limit of Detection)
Li, Na, Mg, K, and Ca: 0.5 pp, by mass
P: 5 ppm by mass
Elements other than the above: 2 ppm by mass
(Minimum limit of quantification)
Li, Na, Mg, K and Ca: 2 ppm by mass
P: 10 ppm by mass
Elements other than the above: 5 ppm by mass

TABLE 3

| | Results of element analysis (ppm by mass) | | | | | | | | | | |
|---|---|---|---|---|---|---|---|---|---|---|---|
| | A1 | B | Ba | Ca | Cd | Co | Cr | Cu | Fe | K | Li |
| Example | 10.4 | ND | ND | ND | ND | ND | 13 | ND | 10.1 | 6.8 | ND |
| 1 | | | | | | | | | | | |
| Example | 8 | ND | ND | ND | ND | ND | 5 | ND | (5) | ND | ND |
| 2 | | | | | | | | | | | |
| Example | 9 | ND | ND | ND | ND | ND | 5 | ND | 6 | ND | ND |
| 3 | | | | | | | | | | | |
| Example | 11 | ND | ND | ND | ND | ND | (3) | ND | 15 | ND | ND |
| 4 | | | | | | | | | | | |
| Example | 10 | 25 | ND | ND | ND | ND | (3) | ND | 17 | ND | ND |
| 5 | | | | | | | | | | | |
| Example | 7 | ND | ND | 2.3 | ND | ND | 5.2 | 45 | 17 | 1.9 | ND |
| 6 | | | | | | | | | | | |
| Example | 13 | ND | ND | ND | ND | ND | (4) | ND | 12 | ND | ND |
| 7 | | | | | | | | | | | |
| Example | 24 | ND | ND | ND | ND | ND | (3) | ND | 19 | ND | ND |
| 8 | | | | | | | | | | | |
| Example | 9 | ND | ND | ND | ND | ND | (3) | ND | 13 | ND | ND |
| 9 | | | | | | | | | | | |
| Example | 15 | ND | ND | ND | ND | ND | ND | ND | ND | ND | ND |
| 10 | | | | | | | | | | | |
| Example | 9 | ND | ND | ND | ND | ND | ND | ND | ND | ND | ND |
| 11 | | | | | | | | | | | |
| Example | 5 | ND | ND | ND | ND | ND | ND | ND | ND | ND | ND |
| 12 | | | | | | | | | | | |
| Example | 15 | ND | ND | ND | ND | ND | 15 | ND | 38 | 6.1 | ND |
| 13 | | | | | | | | | | | |
| Example | 22 | ND | ND | (1.3) | ND | ND | 12 | ND | 12 | 11 | ND |
| 14 | | | | | | | | | | | |
| Example | 15 | ND | ND | (1.9) | ND | ND | 11 | ND | 12 | 12 | ND |
| 15 | | | | | | | | | | | |
| | Results of element analysis (ppm by mass) | | | | | | | | | | |
| | Mg | Mn | Mo | Na | Ni | P | Pb | Sb | Si | Zn | Zr |
| Example | ND | ND | ND | 12.5 | 5.9 | ND | ND | ND | 16.4 | ND | ND |
| 1 | | | | | | | | | | | |
| Example | ND | ND | ND | (1.7) | 7 | ND | ND | ND | ND | ND | ND |
| 2 | | | | | | | | | | | |
| Example | ND | ND | ND | (1.9) | 7 | ND | ND | ND | (2) | ND | ND |
| 3 | | | | | | | | | | | |
| Example | ND | ND | ND | (1.1) | (5) | 14 | ND | 47 | 15 | ND | ND |
| 4 | | | | | | | | | | | |
| Example | (1.1) | ND | ND | 10 | (4) | 110 | ND | ND | 18 | ND | ND |
| 5 | | | | | | | | | | | |
| Example | ND | ND | ND | 11.2 | 5.9 | ND | (2) | (1) | 26 | ND | ND |
| 6 | | | | | | | | | | | |
| Example | ND | ND | ND | (1.9) | (4) | ND | ND | ND | ND | ND | ND |
| 7 | | | | | | | | | | | |
| Example | ND | ND | ND | (0.7) | ND | (6) | ND | ND | ND | ND | ND |
| 8 | | | | | | | | | | | |
| Example | 2.1 | ND | ND | (0.8) | ND | ND | ND | ND | 12 | ND | ND |
| 9 | | | | | | | | | | | |
| Example | 4.1 | ND | ND | 2.2 | ND | (7) | ND | ND | 18 | 14 | 14 |
| 10 | | | | | | | | | | | |
| Example | ND | ND | ND | 3.6 | ND | (6) | ND | ND | ND | ND | ND |
| 11 | | | | | | | | | | | |
| Example | ND | ND | ND | 2.8 | ND | ND | ND | ND | ND | ND | ND |
| 12 | | | | | | | | | | | |
| Example | ND | ND | ND | 12 | 8 | 30 | ND | 38 | (3) | ND | ND |
| 13 | | | | | | | | | | | |
| Example | (0.9) | ND | ND | 9.1 | 5 | ND | ND | ND | 630 | 6 | 30 |
| 14 | | | | | | | | | | | |
| Example | ND | ND | ND | 9.1 | 5 | (6) | ND | ND | 130 | (4) | 38 |
| 15 | | | | | | | | | | | |

1. A method for producing a particle, comprising a step of heating a mixture containing TiH₂ and TiO₂ at 700 to 900° C.,
wherein a molar ratio of the TiH₂ to the TiO₂ contained in the mixture is 3.1 to 4.6.

2. The method according to claim 1, wherein the mixture is heated under an Ar gas atmosphere in the step.

3. A particle having a crystal composition composed of Ti₂O₃ and γ-Ti₃O₅,
wherein a molar ratio of the Ti₂O₃ to the γ-Ti₃O₅ is 0.1 or more.

4. The particle according to claim 3, wherein a* value is 0 or more and b* value is 0 or less in L*a*b* color space.

5. The particle according to claim 3, wherein a total content of Na, K and P in the particle is 2000 ppm by mass or less.

6. A dispersion comprising:
the particle according to claim 3; and
a dispersion medium.
